(19) United States
(12) Patent Application Publication
(10) Pub. No.: US 2023/0160863 A1
(43) Pub. Date: May 25, 2023

(54) METHODS FOR OBTAINING ADSORPTION ISOTHERMS OF COMPLEX MIXTURES

(71) Applicant: EXXONMOBIL TECHNOLOGY AND ENGINEERING COMPANY, Annandale, NJ (US)

(72) Inventors: Yogesh V. JOSHI, Bridgewater, NJ (US); Anantha Sundaram, Annandale, NJ (US); Changyub Peak, Bridgewater, NJ (US); Randall D. PARTRIDGE, Califon, NJ (US); Wenjun Li, Phillipsburg, NJ (US); Carla S. PEREIRA, Bridgewater, NJ (US); Nikki J. Bakas, Doylestown, PA (US)

(21) Appl. No.: 17/920,242

(22) PCT Filed: Feb. 16, 2021
(86) PCT No.: PCT/US2021/018245

§ 371 Date: Oct. 20, 2022

Related U.S. Application Data
(60) Provisional application No. 63/013,204, filed on Apr. 21, 2020.

(51) Int. Cl. G01N 30/86; G16C 20/20; G16C 20/30; G16C 20/70; G01N 30/68; G01N 30/06; G01N 30/46; G01N 33/28; B01D 15/42

(52) U.S. Cl. CPC G01N 30/8693; G16C 20/20; G16C 20/30; G16C 20/70; G01N 30/68; G01N 30/06; G01N 30/463; G01N 33/2835; B01D 15/428; G01N 2030/025

(57) ABSTRACT

The present disclosure provides methods for determining adsorption isotherms for complex mixtures. In at least one embodiment, a method for obtaining adsorption isotherms for liquid mixtures includes providing a column comprising an adsorbent. The method includes delivering a composition to the column, the composition comprising a multi-component feed and a solvent. The method includes collecting a sample from the column and introducing the sample to a two dimensional gas chromatograph to determine a time-series concentration of one or more components of the sample. The method includes integrating the time-series concentration of at least one of the one or more components to determine an isotherm of the at least one component. The method includes obtaining quantitative information of the at least one component, based on the isotherm of the at least one component.

### FIELD

The present disclosure provides methods for obtaining adsorption isotherms of complex mixtures.

### BACKGROUND

Industrial processes in the oil and gas industry include the use of many different feeds, such as refinery feeds (e.g., crude oil), intermediate streams, or refined products. Refinery process conditions (such as temperature, pressure, and/or type of catalyst) are dependent on the chemical composition of a refinery feed. Because the chemical composition of a refinery feed, such as crude oil, is very diverse depending on, for example, where the refinery feed was extracted from across the world, refinery process conditions should be tuned to the type of feed being refined. Such feeds and refinery streams consist of multiple molecular classes as shown in Error! Reference source not found. In order to provide the best conditions for a refinery operation, the disposition for each of the molecular classes in various refinery product or intermediate streams should be determined. For example, a large iso-paraffin molecule is suitable as a lubricant base-stock while a small aromatic molecule is suitable as a gasoline component. Typically adsorptive, extractive, membrane processes can be used to achieve molecular class separation which is typically not possible with traditional distillation based separation. For designing new adsorptive separation processes offering molecular class separation, the inventors of the present disclosure have determined that one needs to quantify and predict adsorption behavior of complex molecular compositions on porous sorbents. In order to quantify adsorption behavior of multiple components in a complex feed using conventional methodology, a multi-year effort would be required. Faster methods to quantify adsorption behavior for complex feeds on any sorbent is needed to aid the novel process design.

In addition, for traditional refining separations, a refinery catalytic process is not just dependent on the chemical components of the feed but also on the concentration of each component of the feed as well as each component's adsorptive interactions with any solid phase components used in a refinery process (such as a zeolite catalyst). For such catalytic process, the models used for process optimization are generally lumped models (e.g. Langmuir-Hinshelwood Rate expression) without explicit description of the adsorption contribution to catalytic rate. This is mainly because a feed can include hundreds or more different molecules, and adsorptive behavior of the different molecules and various mixtures/concentrations of the feed on a solid phase component would need to be determined using thousands of individual adsorption experiments. Accordingly, the labor and time intensive refinery tuning process involves large amounts of refinery feed and time. There are also complexities involved with interpreting the data obtained from such experiments. Because of these cumulative complexities of these process refinery processes and catalytic materials are under optimized.

### SUMMARY

The present disclosure provides methods for determining adsorption isotherms for complex mixtures, as shown in FIG. 14.

In at least one embodiment, a method for obtaining adsorption isotherms for liquid mixtures includes providing a column comprising an adsorbent. The method includes delivering a composition to the column, the composition comprising a multi-component feed component and a solvent. The method includes collecting a sample from the column and introducing the sample to a two dimensional gas chromatography system to determine a time-series concentration of one or more components of the sample. The method includes integrating the time-series concentration of at least one of the one or more components to determine an isotherm of the at least one component. The method includes predicting quantitative isotherm information of the at least one component, based on the isotherm of the at least one other component.

### BRIEF DESCRIPTION OF THE DRAWING

FIG. 1 is a scheme illustrating various molecular classes in a petroleum stream, according to an embodiment.

FIG. 2 is a diagram illustrating HPLC system used to quantify multicomponent elution behavior for multicomponent feed, according to an embodiment.

FIG. 3 is a graph illustrating Frontal Chromatography showing a traditional way to quantify adsorption isotherm using breakthrough experiment, according to an embodiment. The area corresponding to shaded region (adsorption and desorption fronts) are the loading of the solute (toluene in iso-octane) for a given concentration of 1.8 mol/L of toluene in iso-octane.

FIGS. 4A, 4B are graphs illustrating (1) Traditional Frontal Chromatography ran with multiple feed compositions (5% to 100% Toluene in iso-octane) to generate (2) the adsorption isotherm data on right, according to an embodiment. Each point in the plot on the right represents the loading (either adsorption or desorption) for toluene in iso-octane for Silica Gel adsorbent.

FIG. 5 is an elution profile for pulse of toluene (black elution concentration profile with filled circles) in iso-octane (gray elution concentration profile with empty circles) showing the slope of the isotherm (∂qᵢ*/∂cᵢ) vs concentration on y axis, according to an embodiment.

FIG. 6 is a graph comparing isotherm extracted from pulse integration of ∂qᵢ*/∂cᵢ vs cᵢ and BT measurement, according to an embodiment.

FIG. 7 is a graph illustrating concentration vs. time and concentration vs dq/dc for a multicomponent system, according to an embodiment.

FIG. 8A is a comparison of molecules, according to an embodiment.

FIGS. 8B, 8C are graphs illustrating measured binary isotherm data for various compounds using high-throughput ECP, according to an embodiment.

FIGS. 9A, 9B are graphs illustrating experimental vs. simulation of multicomponent elution validating the invented workflow, according to an embodiment.

FIGS. 10A-10H are graphs illustrating (1) an integrated approach used to model a system containing hundreds of components from multi-component pulse experiments and/or (2) track the competitive behavior of each component in a multi-component system when the concentration of all the components are continuously varying (as in an adsorption process separating a multicomponent feed), according to an embodiment.

FIG. 11 is a 2DGC representation of kerosene boiling range refinery stream, according to an embodiment.

FIG. 12 is graphs illustrating isotherm extraction for multi-components in the complex refinery stream (kerosene boiling range); (a) reconstructed elution profiles of homolog series from 2DGC analysis of elution fractions; and (b) extracted isotherms based on molecular classes (paraffins, naphthenes and aromatics), according to an embodiment.

FIGS. 13A, 13B are parity plots extending predictive approach to complex refinery stream, according to an embodiment.

FIG. 14 is a new workflow for predictive liquid adsorption quantification.

### DETAILED DESCRIPTION

### High Performance Liquid Chromatography

In a first process of the present disclosure, a hydrocarbon sample (such as a refinery feed, an intermediate stream, or refined product (such as kerosene, VGO, whole crude, etc.)) is introduced to a high performance liquid chromatograph (HPLC).

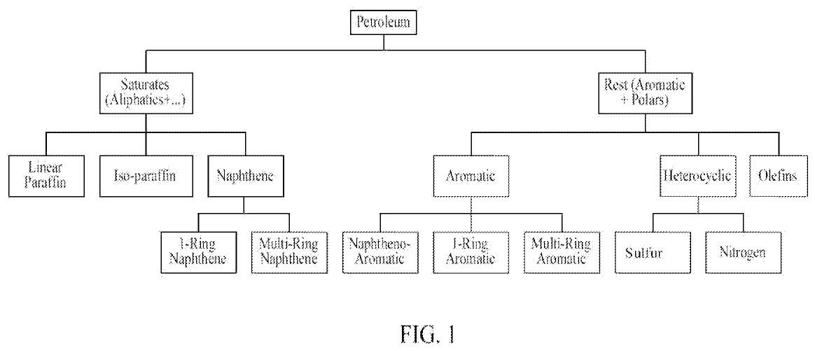

The amount of hydrocarbon sample can have a mass of about 50 mg or greater, or a mass of about 500 mg or greater, or about 1000 mg or greater, or about 5000 mg or greater. A hydrocarbon sample can be optionally collected and one or more HPLC effluents (also referred to as “eluants”) can be combined or separately introduced to an analytical method to understand and quantify the detailed composition of the effluent(s) as function of elution time. The analytical method could include traditional Gas Chromatography, Ultraviolet spectroscopy, Refractive Index Detector (RID), Two Dimensional Gas Chromatography (2DGC) or combination(s) thereof. 2DGC is as described in more detail below. Using HPLC, a hydrocarbon sample can be separated into eluants having different chemical compositions such as an eluant in terms of any of a molecular class (e.g., as shown in FIG. 1). In at least one embodiment, a molecular class is selected from the group consisting of (substituted or unsubstituted) saturates and unsaturates. A saturate can be aliphatic compound(s). In at least one embodiment, saturates include linear paraffin(s), iso-paraffin(s), and/or naphthene(s). Naphthenes may be 1-ring naphthene(s) or multi-ring naphthene(s). Unsaturates can be aromatic hydrocarbon(s), heterocyclic(s), and/or olefins. An aromatic hydrocarbon can be, for example, naphtheno-aromatic(s), 1-ring aromatic(s), and/or multi-ring aromatic(s). A heterocyclic can be, for example, a sulfur-containing heterocyclic or a nitrogen-containing heterocyclic. For example, an eluant fraction can have aromatic hydrocarbons and another eluant fraction can have saturates.

Preparing the sample for separation can include introducing the hydrocarbon sample neat into the HPLC or mixing/dissolving the hydrocarbon sample in an appropriate volume of a suitable organic solvent. The sample can be warmed and agitated to ensure complete miscibility/dissolution with its own components and/or a suitable solvent. Suitable solvents include aromatic or non-aromatic organic solvents, such as hexane, heptane, toluene, cyclohexane, iso-octane, a refinery stream, or combination(s) thereof. The sample or samples can be prepared for separation, such as by dissolution in solvent. In the embodiments disclosed herein, the prepared hydrocarbon sample is a hydrocarbon sample that is dissolved in an appropriate volume of a suitable solvent, as described.

During HPLC, a class of molecules (e.g., having polar compounds such as aromatic hydrocarbons, heterocyclics, and/or olefins) is separated from the prepared hydrocarbon sample. In an exemplary embodiment, separation of a first portion (aromatic hydrocarbons, heterocyclics, and/or olefins) from the prepared hydrocarbon sample proceeds by contacting the prepared hydrocarbon sample with a substrate having preferential affinity for a class of compounds (e.g., aromatic hydrocarbons, heterocyclics, and/or olefins) in the prepared hydrocarbon sample. As embodied herein, a single substrate can selectively (e.g., exclusively) bind a portion (e.g., aromatic portion) in the prepared hydrocarbon sample for elution as an HPLC eluant having a nonpolar portion (e.g., linear paraffins, iso-paraffins, and/or naphthenes) of the hydrocarbon sample.

Any suitable HPLC system can be used. In at least one embodiment, an HPLC system is as shown in FIG. 2. As shown in FIG. 2, a HPLC unit 200 is used to obtain liquid hydrocarbon mixture isotherm information at temperatures, for example, from ambient 20° C. up to 250° C. The adsorbent column(s) 202 that can be used may be placed in an oven 204 operated at the desired temperature, for example 150° C. A preheat coil 206 within the oven can be used to ensure that the solvent/sample is at a desired operating temperature before entering the adsorbent bed (of the column(s) 202) at multiple flow rates. A low dead volume back pressure regulator 208 can be used at the exit of the column(s) 202 maintaining an outlet pressure of, for example, about 40 bar to ensure liquid phase at all temperatures.

A UV detector on the effluent stream can be used for aromatic peak detection at low concentrations. The UV detector could be bypassed if desired. A Fraction Collector 210 can be used with, for example, 2 ml septum capped vials to collect fractions from 0.1 to 1.5 ml, with inserts as needed. Sample collection frequency from 0.25 to 2 minutes can be adjusted to allow accurate reconstruction of the elution profile. Samples can be quantitatively analyzed by gas chromatography and/or 2DGC coupled with flame ionization detectors and appropriate analytical columns.

As embodied herein, separation of saturates and other compounds (e.g., aromatic hydrocarbons, heterocyclics, and/or olefins) from a hydrocarbon mixture can proceed by running the hydrocarbon sample through an HPLC apparatus having one or more chromatography columns containing a substrate having preferential affinity for one or more classes of compounds to be separated (if present in the hydrocarbon sample) such as aromatic hydrocarbons, heterocyclics, olefins, etc. In embodiments where a single column is employed, the chromatography column can contain a substrate with preferential affinity for one or more classes of compounds (e.g., aromatic hydrocarbons, heterocyclics, and/or olefins). Where two or more chromatography columns are employed, one or more of the columns can contain a substrate with preferential affinity for one or more classes of compounds.

As embodied herein, the one or more columns can contain a substrate that exhibits affinity, for example, for aromatic compounds as well as saturate compounds in the presence of a selected solvent or solvent mixture, and exhibits a preferential affinity for aromatic compounds in the presence of another selected solvent or solvent mixture. The aromatic compounds can be selectively eluted from the substrate by contacting the compounds bound on the substrate with a selected solvent or solvent mixture to remove only or substantially only aromatic compounds from the substrate. Alternatively, the one or more columns can contain a substrate that exhibits affinity for aromatic compounds as well as certain saturate compounds in the presence of a selected solvent or solvent mixture, and exhibits a preferential affinity for aromatic compounds in the presence of another selected solvent or solvent mixture. The saturate compounds can be selectively eluted from the column by contacting the compounds bound on the substrate with a selected solvent or solvent mixture, leaving the purified polar compounds bound on the substrate for subsequent elution.

In some embodiments, one column can contain a substrate that exhibits preferential affinity for polar compounds such as unsaturated, multi-ring aromatic hydrocarbons, while a second column can contain a substrate that exhibits preferential affinity for other polar compounds such as single ring aromatic hydrocarbons. By contacting the hydrocarbon sample with the first and second substrates, the hydrocarbon sample can be separated into three fractions, one containing saturated hydrocarbons, one containing single ring aromatic hydrocarbons, and one containing unsaturated, multi-ring aromatic hydrocarbons.

While explicit reference is made herein to first and second columns, it will be readily understood that additional and/or alternative embodiments can employ third, fourth, and additional columns having the same, similar, or different substrates as described for the first and second columns. While explicit reference is made herein to aliphatic, saturates, polar single ring aromatic, polar multi-ring aromatic compounds, it will be readily understood that additional and/or alternative embodiments can apply to any molecular class that is selectively retained on adsorbent in additional columns.

Polar compounds can be purified of nonpolar compounds by selective elution of the polar compounds with one or more solvents. Polar solvents in particular can selectively remove bound polar compounds from the substrate, and the polarity of the solvent or solvent mixture can be selected to selectively remove bound polar compounds based on known or expected strength of binding to the substrate. As embodied herein, the column can be rinsed with a solvent gradient selected to increase or decrease in polarity over the duration of an elution to selectively remove bound compounds (polar or nonpolar) from the substrate. The polar hydrocarbons are concentrated in a polar fraction and the polar fraction can be contaminated with some nonpolar compounds such as saturates and 1-ring aromatic hydrocarbons.

Where reference is made herein to a solvent, it will be understood that “a solvent” can include a single solvent as well as a combination, such as a mixture, of two or more solvents. Similarly, where reference is made to rinsing with a solvent, it will be understood that rinsing can include a single rinse with a single solvent, a single rinse with a combination of two or more solvents, two or more rinses with a single solvent, two or more rinses with two or more separate solvents, two or more rinses with two or more combinations of two or more solvents, etc.

Thus, in exemplary embodiments, the separation can proceed by contacting the prepared hydrocarbon sample with a substrate in a first chromatography column with affinity for a first type of compound (polar, nonpolar, paraffin, isoparaffin, naphthene, etc.). The polar compounds and/or nonpolar compounds of the hydrocarbon sample can be transferred to a second chromatography column. The polar compounds and/or nonpolar compounds of the hydrocarbon sample contact a substrate in the second chromatography column. The substrate in the second chromatography column can exhibit preferential affinity for one or more classes of compounds. The unbound compounds are rinsed from the second column and collected. The bound compounds are then eluted from the substrate exhibiting preferential affinity for the one or more classes of compounds by backflushing the second column with a solvent, such as a polar solvent mixture, and collected. If any residual compounds are left in the second column, the one or more classes of compounds can be selectively eluted by rinsing with a suitable solvent, such as a polar solvent, polar solvent mixture, a nonpolar solvent, and/or nonpolar solvent mixture.

In at least one embodiment, a column has a substrate (also referred to herein as an adsorbent) selected from silica gel, mesoporous organo silica (MOS), a zeolite, a metal organic framework (MOF), zeolitic imidazolate framework (ZIF), or combination(s) thereof.

In at least one embodiment, an HPLC process is performed as a breakthrough (BT) or pulse experiment as follows: (1.1.) Providing a column with an adsorbent material (e.g. Silica Gel Davisil Grade 923). The column volumes can be anywhere between 1 ml to 10 ml, or more. The adsorbent is typically sized to 100 or lower MESH size before packing into the column. (1.2) The Adsorbent column is activated by heating in flowing N₂ for 1 to 3 hr at 100° C. to 200° C. and then transferred to the HPLC system. (1.3) In HPLC setup fill the sample loop (certain volume between 0.1-10 ml) with the solute or solute mixture. (1.4) The HPLC system (tubing, adsorbent filled column, valves, pressure regulators) are flushed with solvent to remove all the preexisting solutes for typically 30-60 minutes at the desired operating temperature, e.g., 150° C. (1.5) In order to do BT or Pulse experiment, a predetermined volume of solute from sample loop is sent through the adsorbent column (1.6) The eluant coming out of the column is collected through a fraction collector with at least 0.1 ml volume per sample. The fraction collection schedule is set in a way to ensure high data density around the adsorption and desorption front. About 10 to 100 samples are collected for each pulse or BT experiment. (1.7) The eluent samples collected are analyzed using GC or 2DGC to quantify elution profile based on weight percentages of solutes in solvents. (1.8) Each HPLC experiment takes about a day including the GC or 2DGC analysis of all the samples.

### Two-Dimensional Gas Chromatography

As mentioned above, for one possible backend characterization, an HPLC eluant is introduced to gas chromatography. For a multicomponent mixture (such as kerosene, VGO, whole crude, etc.), two-dimensional gas chromatography is preferred. Two-dimensional gas chromatography (2DGC or GC×GC) is an analytical separation technique. It can provide high chromatographic resolution of complex mixtures. 2DGC uses a single GC unit containing two separation columns of different selectivity. A modulation unit situated between these two separation columns performs solute focusing and re-injection into a short, high-speed second column.

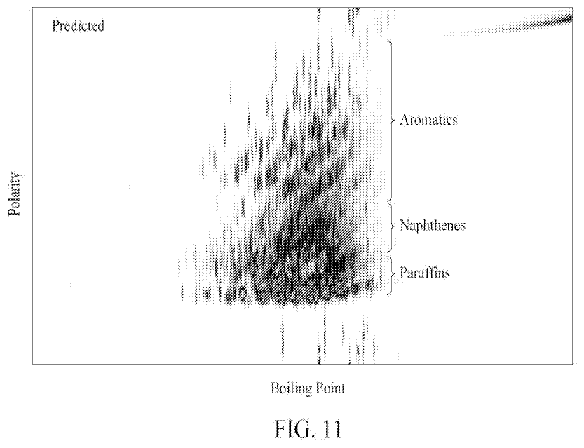

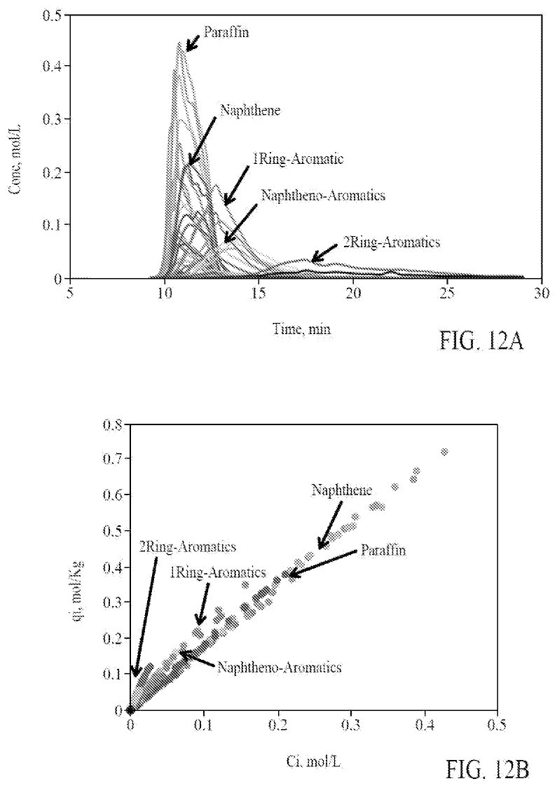

These advances have enabled 2DGC to become an ideal technique for analyzing complex mixtures, such as refinery feeds (and HPLC eluants thereof). One advantage of the 2DGC technique is its enhanced sensitivity due to the re-focusing process during the modulation operation. Another advantage of the 2DGC technique is the qualitative and quantitative analysis through compound class separation. Hence, as shown in FIG. 11, in addition to single component separation, it also provides the compound class homologous series trend information. This trend information can be further combined with the reference standard compounds or corresponding GC-MS (Mass Spectrometry) data to greatly improve the capability of elucidation of individual molecular structure in the complex mixtures.

The 2DGC system can be an Agilent 6890 gas chromatograph (Agilent Technology, Wilmington, Del.) configured with inlet, columns, and detectors. A split/splitless inlet system with a 100 sample position tray auto sampler can be used. The two-dimensional capillary column system can utilize a non-polar first column (e.g., BPX-5, 30 meter, 0.25 mm inner diameter, 1.0 micron film) and a polar second column (e.g., BPX-50, 2 meter, 0.25 mm inner diameter, 0.25 micron film). Both capillary columns can be obtained commercially from SGE Inc. (Austin, Tex.). Loop or thermal modulation systems can be applied. For example, a Zoex thermal modulation assembly (Zoex Corp. Lincoln, Nebr.) is liquid nitrogen or liquid carbon dioxide cooled “trap-release” looped thermal modulator and can be installed between these two columns. Mass spectrometry or a flame ionization detector (FID) can be used for the signal detection or combination of thereof. A feed sample (e.g., 0.2 microliter sample) can be injected with splitless or a split off from about 100:1 to about 1:1 (such as about 50:1 split) at an inlet temperature of from about 200° C. to about 400° C. (such as about 300° C.). Carrier gas flow remains constant or can be ramped based on the head pressure. The head pressure can be programmed from 24 psi with 0-minute hold and 0.2 psi per minute increment to 42 psi with 0-minute hold. The oven can be programmed from about 30° C. with 0-minute hold and about 2.0° C. per minute increment to about 370° C. with 0-minute hold. The hot jet can be programmed from about 150° C. with 0-minute hold and 2.0° C. per minute increment to about 390° C. with a hold time of from about 5-minutes to about 30 minutes, such as about 15-minutes. The total 2DGC run time can be from about 30 minutes to about 2 hours, such as about 90 minutes. The modulation period can be from about 1 second to about 30 seconds, such as about 10 seconds. The sampling rate for the detector can be from about 50 Hz to about 200 Hz, such about 100 Hz.

After data acquisition, the data can be processed for qualitative and quantitative analysis. The qualitative analysis converts data to a two-dimensional image that can be processed by a commercial program (such as GC Image, from GC Image, LLC). The two-dimensional image can be further treated by any suitable program (such as “Photoshop” available from Adobe System Inc. San Jose, Calif.) to generate publication-ready images. Peak volumes can then be quantified.

In at least one embodiment, a two-dimensional chromatographic separation is a combination of non-polar column separation (1st column, X-axis) and polar column separation (2nd column, Y-axis). The non-polar column separation is based on the boiling point of the component in the sample mixture, which closely correlates to the carbon chain length of a component in the feed. It can also be viewed as a boiling point separation. The polar column separation is based on the polarity of the component in the sample mixture, which closely correlates to the functional groups and number of aromatic rings on the component. It can also be viewed as a compound class separation. With this detailed two-dimensional separation, the separated complex mixture can be qualitatively and quantitatively analyzed.

In addition to the qualitative analysis, the 2DGC technique also provides advantages in the quantitative analysis for complex mixtures as compared to conventional GC. Because the 2DGC offers higher resolution for individual components of the feed, better-defined peak integrations thus more accurate quantification of the components are obtained. This improved quantitative analysis gives more accurate compositional information for complex mixtures such as the HPLC eluants of the present disclosure.

An HPLC eluant sample is injected into an inlet device connected to the inlet of a first column to perform a first dimension separation. Sample injection may be by any suitable sample injection device such as a syringe. The sampling device may hold a single sample or may hold multiple samples for injection into the first column. The column can contain a substrate (also referred to as a GC adsorbent) that is usually the column coating material. The first 2DGC column may be coated with a non-polar material. When the column coating material is methyl silicon polymer, the polarity can be measured by the percentage of methyl group substituted by the phenyl group. The polarity of coating materials are measured on a % of phenyl group substitution scale from 0 to 100 with zero being non-polar and 80 (80% phenyl substitution) being considered as polar. These methyl silicon polymers are considered non-polar and have polarity values in the range from 0 to about 20. Phenyl substituted methyl silicon polymers are considered semi-polar and have polarity values of about 21 to about 50. Phenyl substituted methyl silicon polymers coating materials have been called polar materials when greater than 50% phenyl substitution group is included in polymers. Other polar coating polymers, such as carbowaxes, were also used in chromatographic applications. Carbowaxes are high molecular weight polyethylene glycols. In addition, a series of Carborane Silicon polymers sold under the trade name Dexsil have been especially designed for high temperature applications.

The first 2DGC column coated with a non-polar material provides a first separation of one or more classes of compounds of the sample. The first separation, also known as the first dimension, generates a series of bands over a given time period. This first dimension chromatogram is not like the chromatogram that could be obtained from a conventional chromatogram. The bands represent individual components or groups of components of the sample injected, and separated or partially overlapping with adjacent bands. When the complex mixture is separated by the first dimension column, it still has many co-elutions that are not able to be separated by the first dimension column. The bands of separated materials from the first dimension are then sent to the second column to perform a further separation, for example, of the co-eluted components. This further separation is referred to as a second dimension. The second dimension is a second column coated with a semi-polar or polar material, such as a semi-polar coating material.

A modulator manages the flow and separation timing between the end of the first column and the beginning of the second column A modulator may be a thermal modulator that uses a trap/release mechanism. In this mechanism, cold nitrogen or carbon dioxide gas is used to trap a separated sample from the first dimension followed by a periodic pulse of hot nitrogen to release trapped sample to a second dimension. Each pulse is analogous to a sample injection into the second dimension. The role of the modulator is (1) to collect the continuous eluent flow out from the end of the first column with a fixed period of time (modulated period), and (2) to inject collected eluent to the beginning of the second column by releasing collected eluent at the end of modulated period. The function of the modulator is (1) to define the beginning time of a specific second dimensional column separation and (2) to define the length of the second dimensional separation (modulation period). The separated bands from the second dimension are coupled with the bands from the first dimension to form a 2D chromatogram. The bands are placed in a retention plane where the first dimension retention times and the second dimension retention times form the axes of the 2D chromatogram.

In at least one embodiment, Separation column set used can be: 1st Column, SGE BPX-5 (BPX is a phenyl siloxane polymer), 30 meter, 0.25 mm inner diameter, 1.0 micrometer Film and 2nd Column, SGE BPX-50, 3.0 meter, 0.25 mm inner diameter, 0.25 micrometer film. Oven temperature program can be set at 60° C. for 0.0 minutes and ramped at 3.0° C. per minute to 320° C. for 0.0 minutes. The flow program can be constant flow at 2.0 ml per minute for the entire experiment. The inlet temperature can be set at 360° C. with split ratio of 50:1. The sample injection volume can be 0.2 microliter.

Alternatively the separation column set used is: 1st Column, SGE BPX-5 (BPX is a phenyl siloxane polymer), 30 meter, 0.25 mm inner diameter, 1.0 micrometer film, and the 2nd Column, SGE BPX-50, can be 9.0 meter, 0.25 mm inner diameter, and 0.25 micrometer film. The oven temperature program can be set at 170° C. for 0.0 minutes and ramped at 2.0° C. per minute to 390° C. for 0.0 minutes. The flow program can be constant flow at 2.0 ml per minute for an entire experiment. The inlet temperature can be set at 360° C. with split ratio of 50:1, and the sample injection volume can be 0.2 microliter.

### Adsorption Isotherm Data

In order to design an industrial scale separation process the adsorption behavior of a specific chemical compound on given adsorbent should be quantified in a consistent manner so that elution behavior of these compounds at various operating conditions (Temperatures, Pressures and Flow Rates) can be predicted in reliable manner. This is needed since industrial separations process conditions can be different than lab scale HPLC experiments. The consistent way to quantify adsorption behavior is in terms of adsorption isotherm model, which relates the adsorption loading of a component on sorbent phase (qᵢ) to the species concentration in liquid phase.

### Traditional Measurement of the Adsorption Isotherm Using Break-Through (BT) Experiment

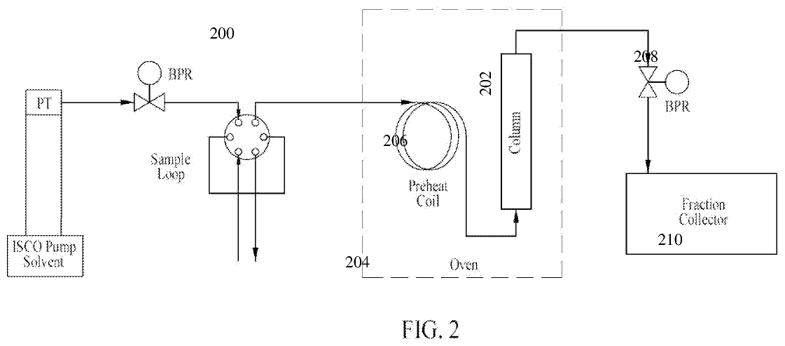

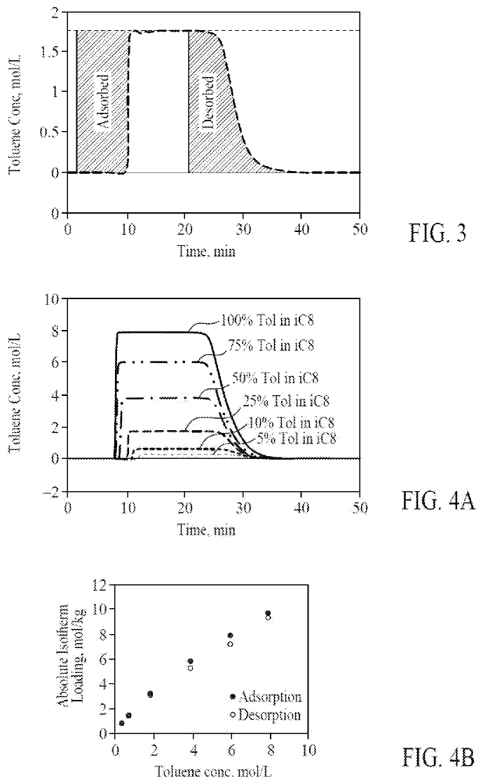

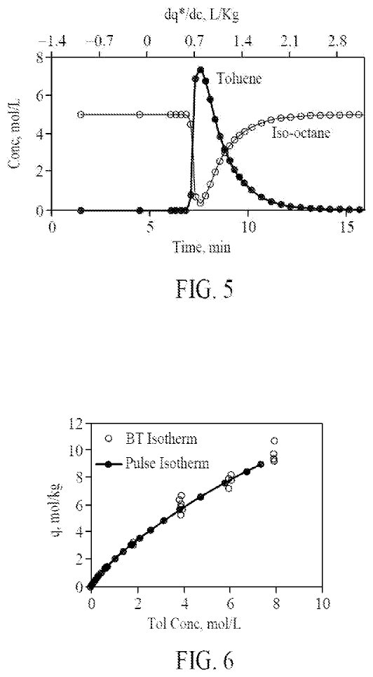

As shown in FIG. 3, the traditional way to quantify adsorption isotherm involves a series of the HPLC experiment where a column packed with the adsorbent of interest (Silica Gel for example) is subjected to a series of compositions of feed compounds. The elution of these compounds is measured using the backend analytical methods such as GC, UV, RI (Refractive Index) depending on the complexity of the feed composition. As shown in FIG. 3, the time delay in the concentration break-though (BT) characterizes the amount of species adsorbed for the given composition (qᵢ*) and shown by shaded region ‘adsorption’. The same area by mass balance can be measured for desorption front (shaded region ‘desorption’). This traditional way of measuring adsorption isotherm, also known as Frontal Analysis, is a slow method to characterize adsorption as one BT experiment gives single point of isotherm data (single composition and corresponding adsorption loading). As shown in FIG. 4, in order to collect the isotherm data, one has to do multiple break-through experiments at multiple feed compositions (for example from 5 to 100 Wt % of Toluene in Iso-octane shown in FIG. 4A). For each concentration breakthrough in elution profile one gets the corresponding loading point (either adsorption or desorption) in FIG. 4B. As the number of components in the feed increases from binary to ternary to multiple components, the composition space grows rapidly. Hence generation of isotherm data through multiple BT experiments to cover the composition space becomes the bottleneck.

### Interpretation and Adsorption Quantification of HPLC Data

Pulse, or BT, volume is first calculated by integrating the area under the elution profile and obtaining the flow rate during the experimental phase. The time reference for the elution characteristic point (ECP), see FIG. 5 and related discussion, to t=0, the time when the feed changes from solute back to solvent. This time reference is determined from the pulse volume and time required for the valve to bring the sample loop inline with, for example, the GC. It is understood that the time axis may be shifted to an earlier time, depending on when the volume of the system (e.g., tubing, valves, joints, pressure regulators, etc.) empties between the sample loop valve and the fraction collector.

Once the time axis is adjusted, the concentration of the elution profile is adjusted to a concentration determined during experimental conditions, by accounting for the temperature dependence of the densities for model compounds. The molar concentration at room temperature, substantially 24 degrees C., is transformed to a molar percentage using room temperature densities. As the molar percentages are constant and invariant of temperature and pressures, the same molar percentages are used for calculating concentrations at experimental conditions (e.g., 150 degrees C.), using temperature dependent pure component densities from Yaw's Handbook. (e.g., ρⱼ^{mol}(T)). In the following equation, xⱼ is the mole fraction of a component in a mixture and ρⱼ^{mol}(T) is the molar density of the pure component at a given temperature:

$${c}_{i} ( T ) = \frac{{x}_{i}}{{Σ}_{j} {x}_{j} / {ρ}_{j}^{mol} ( T )}$$

Once the time axis is adjusted, the slope of the isotherm ∂qᵢ*/∂cᵢ is calculated based on column properties measured during packing of the column and equation shown {Mass transfer, Thomas K. Sherwood, Robert L. Pigford, and Charles R. Wilke, McGraw-Hill Book Company (1975). p 556}. The required column properties, such as bulk density (ρ_{B}) and bulk voidage (E), fluid velocity at experimental temperature (v) and column length (z), as expressed in the following:

$$\frac{\partial {q}_{i}^{*}}{\partial {c}_{i}} = \frac{ε}{{ρ}_{B}} ( \frac{vt}{z} - 1 )$$

The experimental elution profile described above provides data for the cᵢ component at each time (t). Using the method described above, the slope of the isotherm ∂qᵢ*/∂cᵢ at each time t may be calculated. With this calculation, the slope and concentration at each time in the elution profile is determined as shown in Table 1. The slope of the isotherm (3^{rd} column in Table 1 ∂qᵢ*/∂cᵢ) vs concentration (2^{nd} column cᵢ) data can be numerically integrated as shown in the example table below (Table 1), to calculate isotherm information (last column qᵢ*). In some embodiments, the trapezoidal rule is used to carry out numerical integration. The qᵢ and corresponding cᵢ are isotherm data that is used below in developing isotherm models.

TABLE 1

| Time Ref to | | ∂qᵢ^{*} /∂cᵢ (desorption | qᵢ Calculated by |
|---|---|---|---|
| Desorption Front, | Toluene Conc (cᵢ), | tail) using equation in | integrating dq/dc vs |
| min | mol/L @30 C. | 4.6, cm3/g | cᵢ, mol/kg |
| 30.31764105 | 0.025168171 | 3.497492974 | 0.088025502 |
| 27.31764105 | 0.034571251 | 3.106362473 | 0.11907379 |
| 24.31764105 | 0.049658925 | 2.715231972 | 0.162990951 |
| 22.31764105 | 0.063440878 | 2.454478304 | 0.198615302 |
| 21.31764105 | 0.073102263 | 2.32410147 | 0.221699154 |
| 20.31764105 | 0.084647045 | 2.193724637 | 0.247777812 |
| 19.31764105 | 0.098604019 | 2.063347803 | 0.277485736 |
| 18.31764105 | 0.115554662 | 1.932970969 | 0.311355823 |
| 17.56764105 | 0.128522328 | 1.835188344 | 0.335787938 |
| 17.06764105 | 0.139984855 | 1.769999927 | 0.356450222 |
| 16.56764105 | 0.152843127 | 1.70481151 | 0.378790258 |
| 16.06764105 | 0.166564958 | 1.639623093 | 0.401736141 |
| 15.56764105 | 0.18261325 | 1.574434676 | 0.42752621 |

### Multicomponent Isotherm Extraction

A single component (e.g., single solute, single solvent) isotherm extraction is discussed above. Here, the single component solute is replaced by a multi component mixture, for example kerosene, VGO, whole crude, etc.

A pulse or BT experiment similar to that described above is performed for each desired component of the multicomponent mixture. Instead of using the GC for analysis, a 2DGC coupled to an HPLC is used to analyze and quantify the elution behavior of the multicomponent mixture.

Reference time and concentration adjustments are made similar to the single solute experiment described above, lining up the time axes for each measured component of the multicomponent mixture. The isotherm that represents the multicomponent mixture is calculated as follows, in conjunction with the equation shown below: The slope represents the total derivative of the isotherm, that is, the loading of the component “i” qᵢ* is dependent on all concentrations of components in the multi-component mixture. The total derivative in this context provides a collective slope of the isotherm loading coming from changes related to species “i”, and simultaneous change in concentration of other species that occur during elution:

$$\frac{{Dq}_{i}^{*}}{{Dc}_{i}} = \frac{ε}{{ρ}_{B}} ( \frac{vt}{z} - 1 )$$

Although the description of the slope changes as to the total derivative, the integration of this slope with respect to any individual concentration can be done, and provides multicomponent isotherm loading. The integration technique may be the same as discussed above in the single component example (e.g., trapezoidal rule), in some embodiments.

The slope of the isotherm as integrated against the concentration from the elution provides adsorption loading (qᵢ*) for multiple components, at every concentration in the desorption part of the elution profile.

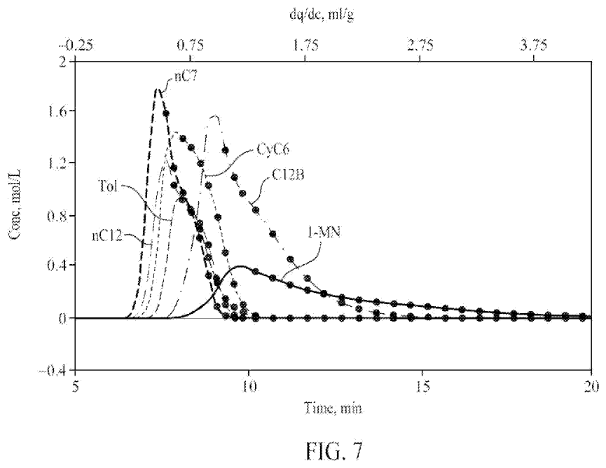

In FIG. 7, an example of a multicomponent mixture elution profile for a six component solute is given, using iso-octane as the solvent. The X-axis on top shows the calculated concentration

$$\frac{{Dq}_{i}^{*}}{{Dc}_{i}}$$

using the equation above. Once

$$\frac{{Dq}_{i}^{*}}{{Dc}_{i}}$$

and corresponding concentrations (shown by dots on the elution profile) are known, the qᵢ* for each component is calculated for each set of concentrations. The table of calculated qᵢ and cᵢ is shown below (Table 2). The missing values are due to zero concentration values for particular species of the multicomponent mixture that provide a zero loading value.

TABLE 2

| | | | | | | |
|---|---|---|---|---|---|---|
| nC7 | nC12 | iC8 | CyC6 | Tol | C12B | 1-MN |

cᵢ @ 150 degree C., mol/L

| | | | | | | |
|---|---|---|---|---|---|---|
| 0 | 0 | 4.993567 | 0 | 0 | 0 | 0.000492 |
| 0 | 0 | 4.993268 | 0 | 0 | 0 | 0.000857 |
| 0 | 0 | 4.993028 | 0 | 0 | 0 | 0.00114 |
| 0 | 0 | 4.992787 | 0 | 0 | 0 | 0.001402 |
| 0 | 0 | 4.992555 | 0 | 0 | 0 | 0.001774 |
| 0 | 0 | 4.991634 | 0 | 0 | 0 | 0.002392 |
| 0 | 0 | 4.991265 | 0 | 0 | 0 | 0.00297 |
| 0 | 0 | 4.990081 | 0 | 0 | 0 | 0.004091 |
| 0 | 0 | 4.989586 | 0 | 0 | 0 | 0.005329 |
| 0 | 0 | 4.988148 | 0 | 0 | 0 | 0.00713 |
| 0 | 0 | 4.986213 | 0 | 0 | 0 | 0.009666 |
| 0 | 0 | 4.98238 | 0 | 0.000472 | 0 | 0.014335 |
| 0 | 0 | 4.977145 | 0 | 0.000559 | 0 | 0.020163 |
| 0 | 0 | 4.974669 | 0 | 0.000758 | 0 | 0.024302 |
| 0 | 0 | 4.970532 | 0 | 0.000823 | 0 | 0.028904 |
| 0 | 0 | 4.965789 | 0 | 0.001174 | 0 | 0.034619 |
| 0 | 0 | 4.960274 | 0 | 0.001511 | 0 | 0.041545 |
| 0 | 0 | 4.954033 | 0 | 0.002455 | 0 | 0.049128 |
| 0 | 0 | 4.947435 | 0 | 0.003196 | 0 | 0.056851 |
| 0 | 0 | 4.938512 | 0 | 0.005232 | 0 | 0.066947 |
| 0 | 0 | 4.930176 | 0 | 0.007967 | 0 | 0.075623 |
| 0 | 0 | 4.92205 | 0 | 0.014339 | 0 | 0.080812 |
| 0 | 0 | 4.910044 | 0 | 0.023 | 0 | 0.088623 |
| 0 | 0 | 4.894361 | 0 | 0.036803 | 0.000219 | 0.098061 |
| 0 | 0 | 4.871973 | 0 | 0.059101 | 0.000267 | 0.108816 |
| 0 | 0 | 4.837376 | 0 | 0.093769 | 0.000645 | 0.124703 |
| 0 | 0 | 4.788954 | 0 | 0.149019 | 0.000456 | 0.142669 |
| 0 | 0 | 4.718818 | 0 | 0.232593 | 0.000823 | 0.16467 |
| 0 | 0 | 4.619319 | 0.00118 | 0.354733 | 0.001628 | 0.192455 |
| 0 | 0.000206 | 4.485411 | 0.001524 | 0.514712 | 0.004261 | 0.229886 |
| 0.000113 | 0.000263 | 4.328663 | 0.00368 | 0.695316 | 0.011351 | 0.269211 |
| 0.001003 | 0.000514 | 4.135919 | 0.04525 | 0.871785 | 0.029727 | 0.300524 |
| 0.008188 | 0.001098 | 3.894649 | 0.206072 | 1.004087 | 0.056853 | 0.303856 |
| 0.041535 | 0.003341 | 3.587651 | 0.451105 | 1.159663 | 0.088362 | 0.26003 |
| 0.155143 | 0.018515 | 3.06283 | 0.796735 | 1.388602 | 0.146305 | 0.183843 |
| 0.399987 | 0.096435 | 2.248756 | 1.178497 | 1.58671 | 0.257691 | 0.107475 |
| 0.697508 | 0.3201 | 1.335569 | 1.527674 | 1.320884 | 0.462745 | 0.061857 |
| 0.898695 | 0.583178 | 0.713193 | 1.788207 | 0.740542 | 0.658542 | 0.034446 |
| 0.999058 | 0.766639 | 0.364137 | 1.872517 | 0.376524 | 0.788012 | 0.015375 |
| 1.09459 | 0.897491 | 0.160293 | 2.024745 | 0.143865 | 0.791384 | 0.001098 |
| 1.261362 | 1.097928 | 0.149252 | 2.097317 | 0.018653 | 0.561859 | 0.001503 |

qᵢ* Calculated, mol/Kg

| | | | | | |
|---|---|---|---|---|---|
| | | | | | 0.003884 |
| | | | | | 0.00666 |
| | | | | | 0.008663 |
| | | | | | 0.010429 |
| | | | | | 0.012828 |
| | | | | | 0.016631 |
| | | | | | 0.020025 |
| | | | | | 0.026282 |
| | | | | | 0.032832 |
| | | | | | 0.041843 |
| | | | | | 0.053796 |
| | | | 0.00209 | | 0.07446 |
| | | | 0.002451 | | 0.098779 |
| | | | 0.003247 | | 0.115304 |
| | | | 0.003495 | | 0.133012 |
| | | | 0.004798 | | 0.154178 |
| | | | 0.005997 | | 0.17883 |
| | | | 0.009219 | | 0.204726 |
| | | | 0.011644 | | 0.229986 |
| | | | 0.018008 | | 0.261549 |
| | | | 0.026165 | | 0.287422 |
| | | | 0.044247 | | 0.302145 |
| | | | 0.067572 | | 0.323184 |
| | | | 0.102757 | 0.000559 | 0.347241 |
| | | | 0.156374 | 0.000673 | 0.373103 |
| | | | 0.234732 | 0.001529 | 0.409012 |
| | | | 0.351637 | 0.001128 | 0.447025 |
| | | | 0.516409 | 0.001852 | 0.490403 |
| | | 0.002156 | 0.739585 | 0.003322 | 0.541171 |
| | 0.000346 | 0.002734 | 1.00881 | 0.007753 | 0.604163 |
| 0.000173 | 0.000435 | 0.006052 | 1.286674 | 0.018661 | 0.664666 |
| 0.001414 | 0.000784 | 0.064008 | 1.532704 | 0.044282 | 0.708322 |
| 0.010524 | 0.001524 | 0.267911 | 1.700446 | 0.078673 | |
| 0.049795 | 0.004167 | 0.556476 | 1.883662 | 0.115781 | |
| 0.175387 | 0.020941 | 0.938566 | 2.136751 | 0.179835 | |
| 0.428388 | 0.101457 | 1.333045 | | 0.294933 | |
| 0.714348 | 0.316431 | 1.668653 | | 0.492018 | |

Both single component isotherm extraction and multicomponent isotherm extraction were used to generate loading vs concentration for the multiple feed components shown above.

### QSAR Model

Quantitative Structure Activity Relationships (QSAR) correlate an activity or property of a molecule or collection of molecules to descriptors that characterize the structure and composition of those molecules. QSAR based adsorption isotherm model relates the adsorption loading to different molecular descriptors of that species and concentration of liquid phase. In hybrid QSAR model a traditional isotherm form is used (e.g. Langmuir) and the isotherm parameters are written as function of the molecular descriptors. In following sections we describe various steps taken to build QSAR based isotherm model.

Molecular Descriptor Pool: Above chemical structures are organized in descriptor databases that take a chemical structure and compute descriptors (Jᵢₖ) e.g., shortest path indices, solubility parameters, structural descriptors such as 1-D, 2-D and 3-D topological indices—Wiener Index, Balaban Index etc.). Known tools and databases Materials Studio and ChemMine are used for building descriptor pool. From these structures, hundreds of structural descriptors may be calculated for all possible feed molecule structures—including the above example, 8 components of the multicomponent mixture.

### Feature/Subset Selection for QSAR Isotherm Model

As a first step of building predictive model, one needs to down-select the important descriptors which are needed for building isotherm model using the isotherm data. Only a small subset of a large pool of potential descriptors is typically relevant to the adsorption isotherm prediction. This step is also called as subset selection. Using the available isotherm data, for each experiment, a dataset of feed descriptors is calculated for all possible molecules using the method described above.

Using a series of linear models, the best subset of feed descriptors that correlates with most of the isotherm data above (qᵢ* vs cᵢ) is selected using regression, or iterative, selection techniques such as LASSO (least absolute shrinkage and selection operator), PCR (Principal component regression), Gaussian Process Regression, Stepwise regression. These descriptors are then used as linear function parameters for functions that use the isotherm data to model the components of a given multicomponent feed. Linear functions are used as they are better suited for dominant factor selection using standard statistical methods (e.g., PCR).

### Predictive Liquid Adsorption

A QSAR hybrid isotherm model is used herein, wherein the full isotherm is given as one of many different isotherm models. In embodiments, a Langmuir single, double, or triple site model may be used:

$${q}_{i}^{*} = \frac{{Q}_{v}}{{v}_{i}} [ \frac{{α}^{1} {K}_{i}^{1} {C}_{i}}{1 + Σ {K}_{j}^{1} {C}_{j}} + \frac{{α}^{2} {K}_{i}^{2} {C}_{i}}{1 + Σ {K}_{j}^{2} {C}_{j}} + \frac{{α}^{3} {K}_{i}^{3} {C}_{i}}{1 + Σ {K}_{j}^{3} {C}_{j}} ]$$

In the Langmuir triple site model above, the superscript indicates the three triple site models. Subscript i indicates the component of a given site of the three triple sites. In the proposed hybrid isotherm model, the Kᵢ¹, Kᵢ², Kᵢ³ are correlated to a molecular descriptor (Jᵢₖ), derived as discussed above. Thus, in this novel hybrid approach, qᵢ* will be correlated to both component molecular descriptor as well as concentration.

In an alternate embodiment, a Freundlich single and/or multi-site model may be used:

$${q}_{i}^{*} = \frac{{Q}_{v}}{{v}_{i}} [ {α}^{1} {{K}_{i}^{1} ( {C}_{i} )}^{1 / n} + {α}^{2} {{K}_{i}^{2} ( {C}_{i} )}^{1 / n} + {α}^{3} {{K}_{i}^{3} ( {C}_{i} )}^{1 / n} ]$$

In the equation above (Freundlich triple site model), adsorption equilibrium constants of the different component are considered as linear function molecular descriptors, selected as described above. This model yields a non-linear hybrid model that may be regressed for the coefficients of non-linear kernels of descriptors that can be directly calculated from the structure of the feed molecules using experimental isotherm data or from several multicomponent experiments.

Kᵢ=g(Jᵢₖ;βₖ)

Where Kᵢ is the parameter from isotherm equations above which is written as nonlinear function (g) of molecular descriptors Jᵢₖ with parameters βₖ.

The above exercise, using either model discussed above, can be repeated for any adsorbent, given experimental data of multicomponent isotherms for that adsorbent, to develop a hybrid isotherm model and its parameters. The above exercise can be carried out using other forms of the adsorption isotherm equations (Langmuir-Freundlich, BET—Brunauer Emmett Teller, Temkin, Everett isotherm).

Utilizing the techniques described above, given an adsorbent, a selection of molecular structural descriptors and isotherm model corresponding to that adsorbent may be determined. From this model, parameters of the isotherm may be calculated from the structures of the feed (and solvent), that is, equilibrium adsorption constants for each molecule in the liquid, including molecules not seen before. The calculated parameters and isotherm model (i.e. one of the hybrid Langmuir and Freundlich models described above) is used to compute the isotherm of every component in the multicomponent feed, using the given adsorbent. The computed isotherm(s) may be used either alone or as part of an adsorption process model to dynamically simulate the separation profile of every component in a multicomponent mixture, using the given adsorbent and solvent. Processes of the present disclosure can reduce the time for determining isotherms of a multicomponent mixture from years down to a day.

### Embodiments

The present disclosure provides, among others, the following embodiments, each of which may be considered as optionally including any alternate embodiments.

Clause 1. A method for obtaining adsorption isotherms for liquid mixtures, the method comprising:

providing a column comprising an adsorbent;

delivering a composition to the column, the composition comprising a multi-component feed and a solvent;

collecting a sample from the column and introducing the sample to a two dimensional gas chromatograph to determine a time-series concentration of one or more components of the sample;

integrating the time-series concentration of at least one of the one or more components to determine an isotherm of the at least one component;

obtaining quantitative information of the at least one component, based on the isotherm of the at least one component.

Clause 2. A method combining a chromatographic adsorptive separation (e.g. HPLC) of a multi-component mixture with advanced analytical techniques (e.g. 2DGC) to measure the detailed molecular composition profile of the effluent.

Dependent Clauses for (1): Variations for separation and Variations of measurement

Specific Clause for (1): HPLC+2DGC
Clause 3. A method to obtain (reliable) multi-component competitive isotherm data based on results of Clause 2.
   a. High-throughput version of (Clause 1) or (Clause 2)
Clause 4. A method to predict the detailed separation profile of a complex liquid mixture for a given adsorbent.
   b. A method to construct a multi-component competitive isotherm model using machine-learning, QSAR and fundamental phenomenological models.
      Clause 5. A method to use (Clause 4) to optimize separation and to determine useful adsorbent and process conditions for desired separation.
      Clause 6. A method for obtaining adsorption isotherms for mixtures, the method comprising:

delivering a composition to a first separation/analytical tool comprising a column having a substrate, the composition comprising a multi-component feed and a solvent; and

collecting a sample from the column and introducing the sample to a second analytical tool to determine a time-series concentration of a components of the sample.

Clause 7. The method of any of Clauses 1 to 6, wherein the second analytical tool is a two-dimensional gas chromatograph.
Clause 8. The method of any of Clauses 1 to 7, wherein a second time-series concentration comprises a tail portion of the time-series concentration qualifying adsorption behavior of components with the given adsorbent.
Clause 9. The method of any of Clauses 1 to 8, further comprising:

using the time-series concentration of at least one of the components to determine slope of adsorption isotherm (total derivative) of the at least one component.

integrating the slope of the isotherm with time-series concentration of at least one of the components to determine an adsorption isotherm of the at least one component.

Clause 10. The method of any of Clauses 1 to 9, comprising

choosing, via a processor, a QSAR attribute for a function corresponding substantially to a logarithm of the tail portion of the time-series concentration exhibiting substantially linear behavior; and

determining via a processor, a composition of the at least one component based upon the chosen QSAR attribute.

Clause 11. The method of any of Clauses 1 to 10, further comprising:

obtaining quantitative information of the component, based on the isotherm of the component.

Clause 12. The method of any of Clauses 1 to 11, wherein the composition comprises a plurality of hydrocarbons.
Clause 13. The method of any of Clauses 1 to 12, wherein the composition is selected from the group consisting of a refinery feed, an intermediate stream, a refined product, and combination(s) thereof.
Clause 14. The method of any of Clauses 1 to 13, wherein the substrate is configured to selectively bind polar compounds.
Clause 15. The method of any of Clauses 1 to 14, wherein the sample comprises a nonpolar compound.
Clause 16. The method of any of Clauses 1 to 15, wherein:

the nonpolar compound is selected from the group consisting of a linear paraffin, an isoparaffin, a naphthene, or combination(s) thereof, and the component is selected from the group consisting of a linear paraffin, an isoparaffin, a naphthene, or combination(s) thereof.

Clause 17. The method of any of Clauses 1 to 16, wherein the substrate is configured to selectively bind nonpolar hydrocarbons.
Clause 18. The method of any of Clauses 1 to 17, wherein the sample comprises a polar compound.
Clause 19. The method of any of Clauses 1 to 18, wherein:

the polar compound is selected from the group consisting of naphtheno-aromatic, 1-ring aromatic, multiring aromatic, a sulfur-containing heterocycle, a nitrogen-containing heterocycle, and combination(s) thereof, and

the component is selected from the group consisting of naphtheno-aromatic, 1-ring aromatic, multiring aromatic, a sulfur-containing heterocycle, a nitrogen-containing heterocycle, and combination(s) thereof.

Clause 20. The method of any of Clauses 1 to 19, wherein introducing the sample to the gas chromatograph is performed using a split inlet system.
Clause 21. The method of any of Clauses 1 to 20, wherein the gas chromatograph comprises a non-polar first column and a polar second column.

Clause 22. The method of any of Clauses 1 to 21, wherein introducing the sample to the gas chromatograph is performed with:

an injection split of from about 100:1 to about 1:1,

an inlet temperature of from about 200° C. to about 400° C.,

a head pressure of from about 24 psi with 0-minute hold and about 0.2 psi per minute increment to about 42 psi with 0-minute hold,

an oven temperature of from about 190° C. with 0-minute hold and about 2.0° C. per minute increment to about 370° C. with 0-minute hold,

a hot jet temperature of from about 240° C. with 0-minute hold and about 2.0° C. per minute increment to about 390° C. with a hold time of from about 5-minutes to about 30 minutes, and

a sampling rate for a detector of from about 50 Hz to about 200 Hz.

Clause 23. The method of any of Clauses 1 to 22, wherein the method further comprises determining the time-series concentration using a flame ionization detector.
Clause 24. The method of any of Clauses 1 to 23, wherein the method further comprises determining the time-series concentration by acquiring data, processing the data by qualitative analysis to convert the data to a two-dimensional image, and processing the two-dimensional image using a program.
Clause 25. The method of any of Clauses 1 to 24, further comprising treating the two-dimensional image with a second program.
Clause 26. The method of any of Clauses 1 to 25, further comprising quantifying peak volumes of the two-dimensional image.
Clause 27. A method for obtaining adsorption isotherms for mixtures, the method comprising:

delivering a composition to a first analytical tool comprising a column having a substrate, the composition comprising a multi-component feed and a solvent; and

collecting a sample from the column and introducing the sample to a second analytical tool to determine a time-series concentration of a component of the sample.

Clause 28. The method of any of Clauses 1 to 27, further comprising a method to construct a multi-component competitive isotherm model, the method comprising:

determining via a processor, an amount of the component adsorbed to an adsorbent based on the time-series concentration of the component;

determining, via a processor, a concentration of the component at equilibrium;

using the time-series concentration of at least one of the components to determine slope of adsorption isotherm (total derivative) of the at least one component.

integrating the slope of adsorption isotherm with time-series concentration of at least one of the one or more components to determine an adsorption isotherm of the at least one component;

determining, via a machine learning model, using a QSAR attributes of feed components along with extracted isotherm data

Clause 29. The method of any of Clauses 1 to 28, wherein the machine learning model comprises:

training a machine learning algorithm to identify isotherm QSAR attributes of potential components of the multi-component feed;

determining, via a processor, coefficients of the components of the machine learning algorithm;

generating the machine learning model based on the machine learning algorithm coefficients; and

predicting, via a machine learning model, the amount of new component adsorbed for a liquid concentration at equilibrium.

Clause 30. The method of any of Clauses 1 to 29, wherein the adsorbent is any of the porous material type, Silica Gel, MOS, Zeolite, MOF, ZIF.
Clause 31. A method for obtaining adsorption isotherms for liquid mixtures, the method comprising:

providing a column comprising an adsorbent;

delivering a composition to the column, the composition comprising a multi-component feed and a solvent;

collecting samples from the column and analyzing the samples with an analytical tool to determine a time-series concentration of one or more components of the sample;

using the time-series concentration of at least one of the components to determine slope of adsorption isotherm (total derivative) of the at least one component.

integrating the slope of adsorption isotherm with time-series concentration of at least one of the one or more components to determine an adsorption isotherm of the at least one component;

obtaining quantitative information of at least one component, based on the adsorption isotherm of the at least one component; and

predicting an isotherm for at least one additional component using data mining and data analytics.

Clause 32. The method of Clause 31, wherein the analytical tool is a gas chromatograph with a flame ionization detector.
Clause 33. The method of Clauses 31 or 32, wherein the analytical tool is a two-dimensional gas chromatograph with a flame ionization detector.
Clause 34. The method of any of Clauses 31 to 33, further comprising using the elution time to calculate a slope of the isotherm called total derivative of isotherm with respect to concentration of at least one component.
Clause 35. The method of any of Clauses 31 to 34, further comprising using a slope of the isotherm along with concentration of at least one component to calculate experimental adsorption loading of at least one component in a liquid mixture.
Clause 36. The method of any of Clauses 31 to 35, wherein obtaining comprises selecting via a processor that includes (a) a selection formulation such as step-wise regression, elastic-net, LASSO applied to (b) a predictor that could be any or a combination of linear models, nonlinear models, ensemble models (such as random forests), black-box models (such as neural networks), or a QSAR feature set that is maximally predictive of the equilibrium partition of the components between the adsorbed and bulk phase.
Clause 37. The method of any of Clauses 31 to 36, wherein obtaining comprises using a processor that includes (i) an adsorption isotherm formulation and its parameters expressed as some linear or nonlinear function of chosen descriptors and (ii) an optimization model which can be linear, nonlinear, discrete or black-box, that estimates a linear or nonlinear relationship in (i) for minimizing the error between the predicted isotherm via (i) and the measured isotherm from multicomponent adsorption experiments.
Clause 38. The method of any of Clauses 31 to 37, wherein the composition comprises a plurality of hydrocarbons.
Clause 39. The method of any of Clauses 31 to 38, wherein the composition is selected from the group consisting of a refinery feed, an intermediate stream, a refined product, and combination(s) thereof.
Clause 40. The method of any of Clauses 31 to 39, wherein the adsorbent is configured to selectively bind polar compounds.
Clause 41. The method of any of Clauses 31 to 40, wherein the sample comprises a nonpolar compound.
Clause 42. The method of any of Clauses 31 to 41, wherein:

the nonpolar compound is selected from the group consisting of a linear paraffin, an isoparaffin, a naphthene, or combination(s) thereof, and

the component is selected from the group consisting of a linear paraffin, an isoparaffin, a naphthene, or combination(s) thereof.

Clause 43. The method of any of Clauses 31 to 42, wherein the adsorbent is configured to selectively bind nonpolar hydrocarbons.

### EXAMPLES

### Example 1: Getting Reliable Isotherm Data from Fewer Experiments (High-Throughput Measurement of Adsorption Isotherm for Multiple Molecular Class Species)

In this example, feed model compound mixture consisting of 12.7 wt. % n-heptane (nC7), 18.4 wt. % n-dodecane (nC12), 19.5 wt. % cyclohexane (CyC6), 21.5 wt. % toluene (Tol), 15.5 wt. % n-dodecylbenzene (C12B), and 12.4 wt. % 1-methylnaphthalene (1-MN) was subjected to HPLC experiment to obtain the component isotherms. A mesoporous organosilica (MOS) adsorbent (as described in U.S. Ser. No. 10/435,514 Calabro, et al) was pelleted and sized to 100-200 mesh and packed in a 250 mm×4.6 mm ID HPLC column resulting in 1.68 g in 4.15 cc after drying at 150° C. Isooctane (2,2,4-trimethylpentane) solvent flow was nominally 0.4 ml/min. at 40 Bar, with the column at 150° C. A 1.0 ml pulse of the hydrocarbon mixture was introduced by the sample valve. Fraction of the column effluent were collected at time intervals from 0.25 to 1.0 minutes and analyzed by gas chromatography (GC) using a Agilent 30 m DB-5 column temperature programmed from 50-250° C. and a flame ionization detector. Weight fractions were calculated using response factors for each component. The reconstructed multicomponent elution is shown in FIG. 7.

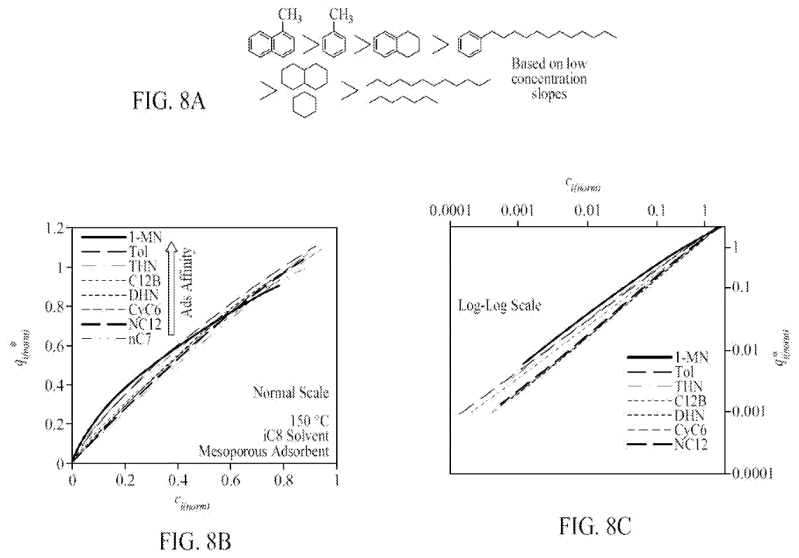

The ECP technique is used in high-throughput mode to measure isotherm for multiple solutes types in iso-octane (Toluene in isooctane; 1-methylnaphthalene in isooctane; Tetrahydronaphthalene in isooctane; Dodecylbenzene in isooctane; Decahydronaphthalene in isooctane; Cyclohexane in isooctane; n-Dodecane in isooctane; n-Heptane in isooctane) to quantify the binary adsorption behavior over three orders of magnitude variation in concentration. The experimental conditions for these binary HPLC experiments is similar as described above and isotherms extracted using the extraction techniques described before. For a given adsorbent like Silica Gel, MOS, these adsorption behavior is mainly the function of the polarity of the solute molecules. A similar experiment can be carried out on other adsorbents to quickly quantify the binary adsorption behavior, thus providing much needed information for data driven QSAR model. As shown in the FIG. 8, the measured isotherm for model compounds shows the higher affinity of the multi-ring aromatics for adsorption compared to saturate molecules like paraffins and cyclo-paraffins.

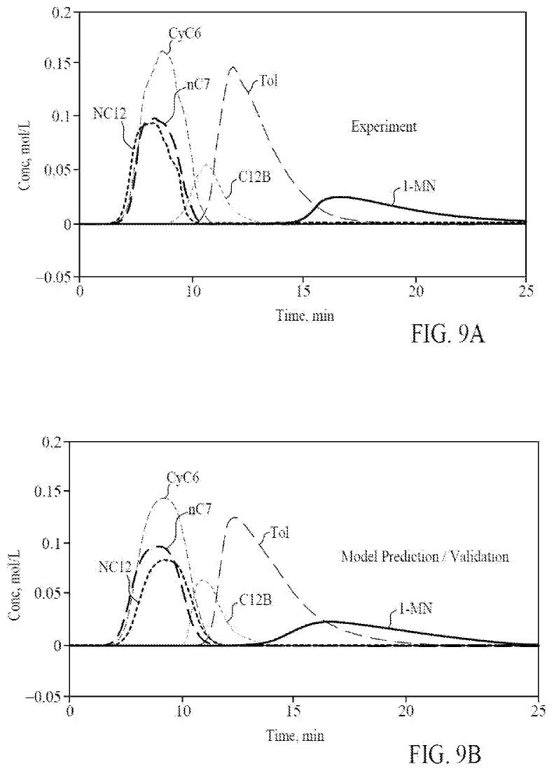

In order to show this high throughput technique to measure the multicomponent isotherm, multiple validation tests were done. Validity of the isotherm measured using ECP technique was tested by optimizing a hybrid QSAR Langmuir isotherm model based on measured isotherm data. The isotherm model was then used in a simulation to predict the multicomponent elution of a 6 component mixture in iso-octane. The simulation output is compared with the experiment using HPLC at same condition to validate the predictive isotherm model, as shown in FIG. 9. The close agreement between the experimental multicomponent elution and model HPLC behavior confirms the validity and novelty of the approach.

### Example 2: Prediction of the Unknown Compound Isotherms Using QSAR

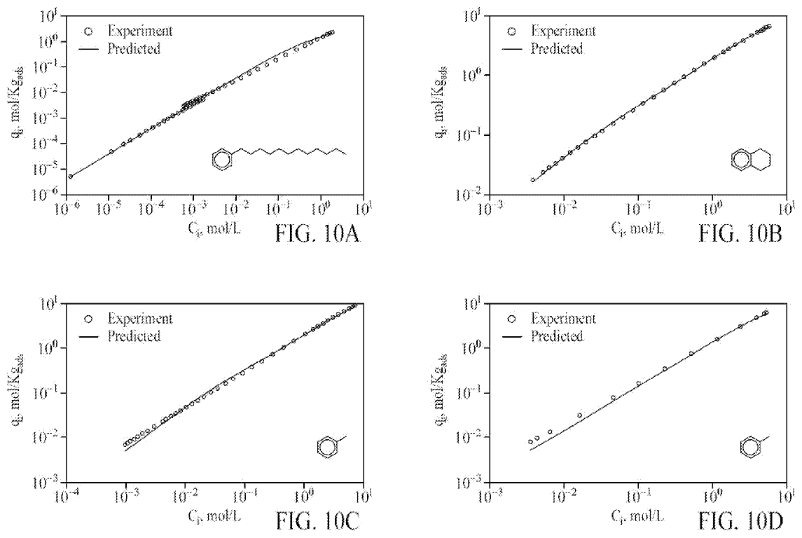

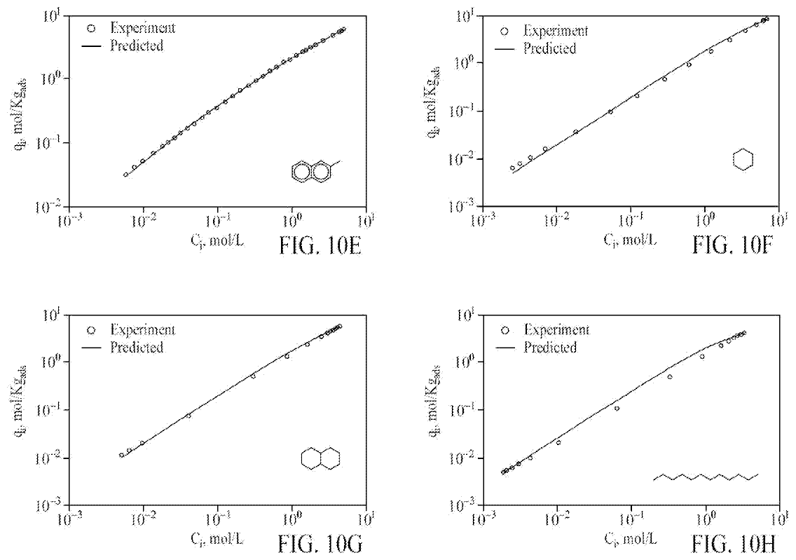

The QSAR approach described above was used in this example to show the validity when applied to the model compounds. In this example, the isotherm data for model compounds was used to learn (variable selection+parameter estimation) the QSAR model to predict the adsorption isotherm of the compounds not part of the learning process. Here an alkyl-aromatic homologous series was used to predict isotherm data. The integrated approach was used to model a system containing hundreds of components from multi-component pulse experiments. FIGS. 10A-H illustrate some typical results. The model tracks the competitive behavior of each component in a multi-component system when the concentration of all the components are continuously varying (as in an adsorption process separating a multicomponent feed). FIGS. 10A-H illustrate results of the estimation. The approach can predict over 4 orders of magnitude in terms of the adsorbed concentrations. Again this is typical of multi-component systems but hitherto not successfully modeled or validated by any of the current approaches.

### Example 3: Measurement of Multicomponent Isotherm for A Complex Mixture in Single Elution Experiment

In this example, a commercial hydrotreated kerosene (Varsol 80) feed was subjected to HPLC experiment to obtain the component isotherms. A mesoporous organosilica (MOS) adsorbent (as described in U.S. Ser. No. 10/435,514 Calabro, et al) was pelleted and sized to 100-200 mesh and packed in a 250 mm×4.6 mm ID HPLC column resulting in 1.68 g in 4.15 cc after drying at 150° C. Isooctane (2,2,4-trimethylpentane) solvent flow was nominally 0.4 ml/min. at 40 Bar, with the column at 150° C. A 1.0 ml pulse of the complex kerosene hydrocarbon mixture was introduced by the sample valve. Fractions of the column effluent were collected at time intervals from 0.25 to 1.0 minutes and analyzed by 2D-GC. Weight fractions were calculated using response factors for each component identified.

In this example, the multicomponent isotherm extraction approach described was applied to a complex multicomponent mixture containing hundreds of species. 2DGC was used for direct measurement of the composition of the mixture of 100s of species. In this example we used 2DGC for analyzing multiple elution samples out of an adsorbent column to quantify the structure-property driver of separation through a QSAR approach. The elution profiles can be interpreted as multicomponent adsorption with 100's of species adsorbed together. The isotherm extraction process described for binary elution profile, can be applied to 100's of elution components together to extract multicomponent isotherm data from single HPLC experiment. The extracted multicomponent isotherm data is shown in FIGS. 11 and 12. The multicomponent isotherm data out of such experiment is effectively parameter estimated using a QSAR based isotherm modeling approach.

### Example 4: Building Predictive Isotherm Models Using a Large Set of Multicomponent (Complex Mixtures) Adsorption Isotherm Data

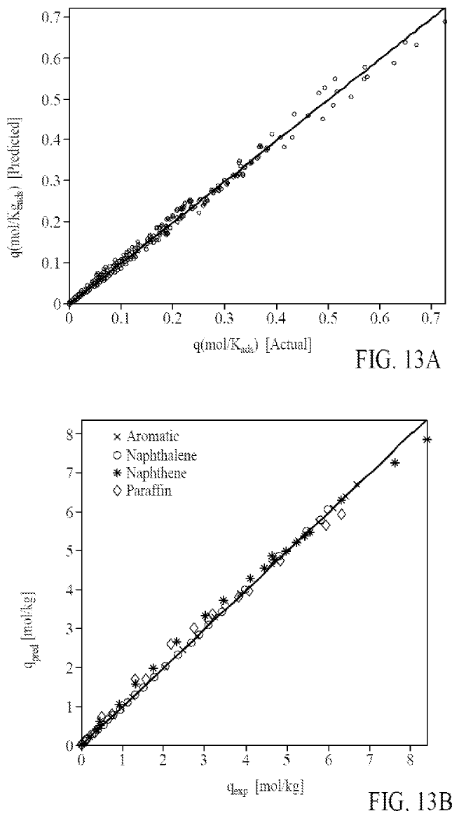

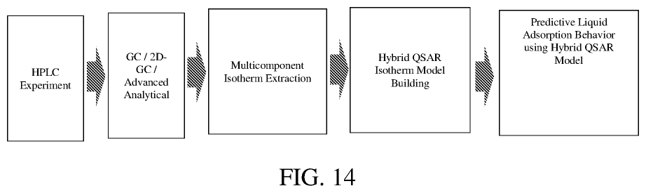

In this example we apply QSAR isotherm building workflow to a much larger isotherm dataset generated using example 3. The objective is to predict the isotherm (or separation characteristic) of a complex target feed with a number of known as well as new components. In order to prove the objective, the components from the Varsol 80 were randomly assigned to two categories: ‘known components’ and ‘new components’. Known component is a molecule whose separation behavior or isotherm is already reasonably characterized. A new component is one whose structure is known but its adsorption isotherm is not well characterized or unknown. As described in Example 3, a multi-component feed experiment with Varsol 80 (hydrotreated kerosene) was carried out to generate thousands of isotherm loading for a complex mixture. This would result in a large compendium of extracted multicomponent isotherm data that looks like a large multiple of a dataset produced from Example 2. The second step is to propose based on the target adsorbent the most likely isotherm model (such as a two-site or three-site Langmuir or a Freundlich isotherm). Using the data-driven and QSAR methodologies described earlier and in Example 2 we build a descriptor based isotherm model (the best structural descriptors, feature transforms and correlating relationships to the parameters of the chosen isotherm functional form). Just using the composition of key structural fragments in the target feed, this isotherm model can predict the competitive adsorption profile of every molecule or lump in the feed. In order to validate the approach only ‘known components’ were used to build the QSAR based hybrid model. This model was used to predict the isotherm loading for the ‘new components’ from the complex mixture. The parity plot for these predictions are shown in the FIG. 13.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures to the extent they are not inconsistent with this text. As is apparent from the foregoing general description and the specific embodiments, while some embodiments have been illustrated and described, various modifications can be made without departing from the spirit and scope of the disclosure. Accordingly, it is not intended that the disclosure be limited thereby. Likewise, the term “comprising” is considered synonymous with the term “including.” Likewise whenever a composition, an element or a group of elements is preceded with the transitional phrase “comprising”, it is understood that we also contemplate the same composition or group of elements with transitional phrases “consisting essentially of,” “consisting of”, “selected from the group of consisting of,” or “is” preceding the recitation of the composition, element, or elements and vice versa.

While the present disclosure has been described with respect to a number of embodiments and examples, those skilled in the art, having benefit of this disclosure, will appreciate that other embodiments can be devised which do not depart from the scope and spirit of the present disclosure.

1. A method for obtaining adsorption isotherms for liquid mixtures, the method comprising:
providing a column comprising an adsorbent;
introducing a composition to the column, the composition comprising a multi-component feed and a solvent;
collecting samples from the column and analyzing the samples with an analytical tool to determine a time-series concentration of one or more components of the sample;
integrating the time-series concentration of at least one of the one or more components to determine an adsorption isotherm of the at least one component;
obtaining quantitative information of at least one component, based on the adsorption isotherm of the at least one component; and
predicting an isotherm for at least one additional component using data mining and data analytics.

2. The method of claim 1, wherein the analytical tool is a gas chromatograph with a flame ionization detector.

3. The method of claim 1, wherein the analytical tool is a two-dimensional gas chromatograph with a flame ionization detector.

4. The method of claim 1, further comprising using the elution time to calculate a slope of the isotherm called total derivative of isotherm with respect to concentration of at least one component.

5. The method of claim 1, further comprising using a slope of the isotherm along with concentration of at least one component to calculate experimental adsorption loading of at least one component in a liquid mixture.

6. The method of claim 1, wherein the obtaining comprises selecting via a processor that includes (a) a selection formulation such as step-wise regression, elastic-net, LASSO applied to (b) a predictor that could be any or a combination of linear models, nonlinear models, ensemble models (such as random forests), black-box models (such as neural networks), or a QSAR feature set that is maximally predictive of the equilibrium partition of the components between the adsorbed and bulk phase.

7. The method of claim 1, wherein the obtaining comprises using a processor that includes (i) an adsorption isotherm formulation and its parameters expressed as some linear or nonlinear function of chosen descriptors and (ii) an optimization model which can be linear, nonlinear, discrete or black-box, that estimates a linear or nonlinear relationship in (i) for minimizing the error between the predicted isotherm via (i) and the measured isotherm from multicomponent adsorption experiments.

8. The method of claim 1, wherein the composition comprises a plurality of hydrocarbons.

9. The method of claim 8, wherein the composition is selected from the group consisting of a refinery feed, an intermediate stream, a refined product, and combination(s) thereof.

10. The method of claim 1, wherein the adsorbent is configured to selectively bind polar compounds.

11. The method of claim 10, wherein the sample comprises a nonpolar compound.

12. The method of claim 11, wherein:
the nonpolar compound is selected from the group consisting of a linear paraffin, an isoparaffin, a naphthene, or combination(s) thereof, and
the component is selected from the group consisting of a linear paraffin, an isoparaffin, a naphthene, or combination(s) thereof.

13. The method of claim 1, wherein the adsorbent is configured to selectively bind nonpolar hydrocarbons.

14. A method for obtaining adsorption isotherms for mixtures, the method comprising:
introducing a composition to a first analytical tool comprising a column having a substrate, the composition comprising a multi-component feed and a solvent; and
collecting a sample from the column and introducing the sample to a second analytical tool to determine a time-series concentration of a component of the sample.

15. The method of claim 14, further comprising a method to construct a multi-component competitive isotherm model, the method to construct comprising:
determining, via a processor, an amount of the component adsorbed to an adsorbent based on the time-series concentration of the component;
determining, via the processor, a concentration of the component at equilibrium;
calculating the amount of component adsorbed to the adsorbent and concentration of the component at equilibrium; and
determining, via a machine learning model, an isotherm for the component using the calculated amount of component adsorbed for a measured concentration at equilibrium.

16. The method of claim 15, wherein the machine learning model comprises:
training a machine learning algorithm to identify specific isotherm quantitative structure activity relationship (QSAR) attributes of potential components of the multi-component feed;
determining, via a processor, one or more descriptors of the components of the machine learning algorithm;
generating the machine learning model based on the machine learning algorithm; and
predicting, via a machine learning model, the amount of new component adsorbed for a liquid concentration at equilibrium.

17. A method for predicting adsorption isotherms for liquid mixtures, the method comprising:
providing a column comprising an adsorbent;
delivering a composition to the column, the composition comprising a multi-component feed and a solvent;
collecting a sample from the column and introducing the sample to a two dimensional gas chromatograph to determine a time-series concentration of one or more components of the sample;
integrating the time-series concentration of at least one of the one or more components to determine a isotherm of the at least one component;
predicting quantitative information of the at least one component, based on the isotherm model of the at least one component.
